SEQUENCE LISTING (13 sequences)

SEQ ID NO: 1 - DNA, 19 bp, Artificial Sequence
  A synthetic oligonucleotide
gacttcgagcaggagatgg

SEQ ID NO: 2 - DNA, 26 bp, Artificial Sequence
  A synthetic oligonucleotide
gcacggtgttggtgttggcgtagagg

SEQ ID NO: 3 - DNA, 23 bp, Artificial Sequence
  A synthetic oligonucleotide
agccatcgctacctgaaacatgc

SEQ ID NO: 4 - DNA, 23 bp, Artificial Sequence
  A synthetic oligonucleotide
atcatgaaggcacaggcttccag

SEQ ID NO: 5 - DNA, 20 bp, Artificial Sequence
  A synthetic oligonucleotide
tgcaaagagggttgtcagag

SEQ ID NO: 6 - DNA, 21 bp, Artificial Sequence
  A synthetic oligonucleotide
aagagacaaggtgagctgaag

SEQ ID NO: 7 - DNA, 24 bp, Artificial Sequence
  A synthetic oligonucleotide
tcagccagagccctataaatgctg

SEQ ID NO: 8 - DNA, 22 bp, Artificial Sequence
  A synthetic oligonucleotide
tcttcctgcactctgtccaagc

SEQ ID NO: 9 - DNA, 21 bp, Artificial Sequence
  A synthetic oligonucleotide
ggattaagggacctgttacag

SEQ ID NO: 10 - DNA, 21 bp, Artificial Sequence
  A synthetic oligonucleotide
gcaaatacttcacttggcctg

SEQ ID NO: 11 - protein, 10 aa, Artificial Sequence
  A synthetic peptide
EQKLISEEDL

SEQ ID NO: 12 - protein, 8 aa, Artificial Sequence
  A synthetic peptide
DYKDDDDK

SEQ ID NO: 13 - protein, 8 aa, Artificial Sequence
  A synthetic peptide
WSHPQFEK

(19) United States
(12) Patent Application Publication
(10) Pub. No.: US 2013/0316022 A1
(43) Pub. Date: Nov. 28, 2013

(54) METHOD TO ENHANCE ANTIMICROBIAL ACTIVITY BY INCREASING pH

(71) Applicant: University of Iowa Research Foundation, Iowa City, IA (US)

(72) Inventors: Michael J. Welsh, Riverside, IA (US); Joseph Zabner, Iowa City, IA (US)

(73) Assignee: University of Iowa Research Foundation, Iowa City, IA (US)

(21) Appl. No.: 13/871,594

(22) Filed: Apr. 26, 2013

Related U.S. Application Data
(60) Provisional application No. 61/639,743, filed on Apr. 27, 2012.
(60) Provisional application No. 61/653,483, filed on May 31, 2012.

(51) Int. Cl. A61K 33/00

(52) U.S. Cl. CPC A61K 33/00
USPC 424/715

(57) ABSTRACT

The invention provides a method to enhance antimicrobial activity, e.g., in a mammal.

### CROSS-REFERENCE TO RELATED APPLICATIONS

The application claims the benefit of the filing date of U.S. application Ser. No. 61/653,483, filed on May 31, 2012, and U.S. application Ser. No. 61/639,743, filed on Apr. 27, 2012, the disclosures of which are incorporated by reference herein.

### BACKGROUND

Current methods of preventing and treating infections are inadequate, and bacteria have developed resistance to pharmaceutical antibiotics. Many organisms, including vertebrates such as humans, produce antimicrobials (examples are defensins, lysozyme, lactoferrin, and the like) that kill bacteria. Those native (endogenous) antimicrobials cover body surfaces that are exposed to the environment, e,g., the pulmonary airways, skin, cornea, and intestine. In contrast to current pharmaceutical antibiotics, bacteria have not developed resistance to endogenous antimicrobials.

### SUMMARY OF THE INVENTION

The present invention provides methods to enhance the activity of antimicrobials, either those that are naturally produced by an animal, such as an avian or mammal, e.g., a human, ovine, bovine, equine, swine, canine, feline and the like. As described herein below, in newborn wild-type pigs, the thin layer of airway surface liquid (ASL) rapidly killed bacteria in vivo, when removed from the lung, and in primary epithelial cultures. Lack of cystic fibrosis transmembrane receptor (CFTR) reduced bacterial killing. ASL pH was found to be more acidic in cystic fibrosis (CF), and reducing pH inhibited the antimicrobial activity of ASL. Reducing ASL pH also diminished bacterial killing in wild-type pigs, and increasing ASL pH rescued killing in CF pigs. These results directly link the initial host defense defect to loss of CFTR, an anion channel that facilitates HCO₃⁻ transport. Without CFTR, airway epithelial HCO₃⁻ secretion is defective, ASL pH falls and inhibits antimicrobial function, and thereby impairs killing of bacteria that enter the newborn lung. Thus, a reduced pH in airway surface liquid inhibits the activity of antimicrobials, and increasing airway surface liquid pH enhances bacterial killing in the lungs. These findings indicate that increasing pH may enhance the antimicrobial activity of endogenous antimicrobials, as well as non-naturally occurring antimicrobials (i.e., pharmaceutical antibiotics which those that are not encoded by the genome of an animal such as a vertebrate) and thereby prevent infection and/or treat infection in lungs as well as at other body surfaces including skin, cornea and intestine.

Solutions, e.g., buffers, reagents and excipients to increase pH may be added to any solution or cream applied to an external or internal body surface to increase aggregate antimicrobial activity. As an example, the pH of solutions, creams, etc. that contain pharmaceutical antibiotics may be increased in order to enhance the additive or synergistic effects of the administered antibiotic and the endogenous antimicrobial. In addition, the pH of solutions or creams that possess other antimicrobial activity, for instance, solutions with low ionic strength, solutions containing SCN or iodide and the like, may be elevated in order to enhance bacterial killing activity. In one embodiment, a composition of the invention such as a cream or aqueous solution, e.g., having buffers, reagents and excipients to increase pH, increase the endogenous (native) pH of a body surface by at least 0.1, 0.2, 0.3, 0.4, 0.5, 0.6, 0.7, 0.8, 0.9, 1.0, or more pH units. In one embodiment, the pH of lung epithelium is increased by about 0.1 pH units to about 1 pH units after administration of a composition of the invention. In one embodiment, the pH of lung epithelium is increased by about 0.5 pH units to about 0.8 pH units after administration of a composition of the invention. Reagents that increase pH include but are not limited to buffers such as HEPES, TRIS, acetate and HCO₃⁻. Other reagents include those that inhibit H⁺-ATPases, e.g., H2 receptor antagonists such as cimetidine, or proton pump inhibitors such as omeprazole, Na⁺—H⁺ exchangers, e.g., desipramine, or stimulators of Na⁺—HCO₃⁻ transporters, e.g., dopamine.

Thus, the invention provides a method to prevent, inhibit or treat microbial infection in an avian or mammal. The method includes administering to an avian or mammal having a microbial infection or suspected of being exposed to a microbe an effective amount of a composition having one or more reagents that increase pH.

In one embodiment, the invention provides a method to prevent, inhibit or treat microbial infection in an avian or mammal. The method includes selecting a composition having one or more reagents that increase pH suitable to increase pH of a body surface of an avian or mammal when applied thereto; and administering to an avian or mammal having a microbial infection or suspected of being exposed to a microbe an effective amount of a composition having one or more reagents that increase pH.

In one embodiment, the composition is administered to an epithelial surface. In one embodiment, the surface is the skin, the cornea. or the intestinal epithelium. In one embodiment, the composition is administered as an aerosol. In one embodiment, the composition is administered as liquid drops. In one embodiment, the composition is a cream. In one embodiment, the composition is orally administered. In one embodiment, the composition is a sustained release composition. In one embodiment, the composition comprises a buffer. In one embodiment, the buffer comprises HCO₃⁻, acetate, HEPES, or TRIS. In one embodiment, the one or more reagents inhibit proton secretion. In one embodiment, the reagent inhibits H⁺-ATPases, Na⁺—H⁺ exchangers, H⁺-conducting ion channels, or other transporters or channels. In one embodiment, the one or more reagents stimulate secretion of a base. In one embodiment, one reagent stimulates secretion of HCO₃⁻. In one embodiment, one reagent stimulates channels that conduct bases. In one embodiment, one reagent stimulates Na⁺—HCC₃⁻ transporters. In one embodiment, the one or more reagents stimulate H⁺ absorption. In one embodiment, the one or more reagents inhibit HCO₃⁻ or other base absorption. In one embodiment, a plurality of reagents that increase pH is employed. In one embodiment, the microbe is a bacterium, e.g., a gram negative bacterium or a gram positive bacterium. In one embodiment, the mammal is a human. In one embodiment, the mammal has a defect in CFTR.

Further provided is a support to detect antimicrobial activity in vivo, comprising: a biocompatible support having a region or a linear or two-dimensional microarray of discrete regions, each having a defined area, formed on an upper surface of the support, each region comprising a covalently linked ligand which is bound to a receptor for the ligand, which receptor is covalently linked to a microbe. In one embodiment, the microbe is a bacteria or a fungus. In one embodiment, the biocompatible support comprises metal. In one embodiment, at least two of the discrete regions have a different microbe. In one embodiment, at least one of the discrete regions has a drug resistant microbe. In one embodiment, the support includes a cover. In one embodiment, the ligand comprises avidin or streptavidin. In one embodiment, the receptor comprises biotin. In one embodiment, the ligand comprises an antibody or an antigen binding portion thereof. In one embodiment, the receptor comprises an antibody or an antigen binding portion thereof. In one embodiment, the ligand or receptor comprises a His tag, C-myc tag (EQKLISEEDL), Flag tag (DYKDDDDK), SteptTag (WSHPQFEK), GST, thioredoxin, cellulose binding domain, chitin binding domain, metal binding domain, e.g., zinc binding domains or calcium binding domains such as those from calcium-binding proteins, e.g., calmodulin, or maltose binding protein. In one embodiment, the bacteria is a Streptococcus, E. coli or Staphylococcus.

The invention provides a method to detect antimicrobial activity in vivo. The method includes providing the suppor; introducing the support to an animal for a period of time; removing the support from the animal; and detecting whether the microbe in one or more of the defined areas is viable or has an altered property. In one embodiment, the animal is a mammal. In one embodiment, the mammal is a human, ovine, bovine, equine, feline, canine, or swine. In one embodiment, in the support is introduced to the pulmonary airway, skin, cornea, or intestine of the animal. In one embodiment, the altered property is relative to a corresponding microbe that is on a support but not introduced to the animal. In one embodiment, the support is introduced to a mucosal surface of the animal. In one embodiment, antimicrobial activity is determined at the site of introduction. In one embodiment, a decrease in antimicrobial activity relative to a control or over time is indicative of an immune deficiency. In one embodiment, the animal is administered antimicrobial therapy. In one embodiment, the altered property is a metabolic response.

Also provided is a method to detect antimicrobial activity in vivo, comprising: providing the support; introducing the support to an animal for a period of time; and detecting in vivo whether the microbe in one or more of the defined areas is viable or has an altered property. In one embodiment, the microbe is a bacteria or fungus. In one embodiment, the biocompatible support comprises metal. In one embodiment, at least two of the discrete regions have a different microbe. In one embodiment, at least one of the discrete regions has a drug resistant microbe. In one embodiment, the ligand comprises avidin or streptavidin. In one embodiment, the receptor comprises biotin. In one embodiment, the ligand comprises an antibody or an antigen binding portion thereof. In one embodiment, the receptor comprises an antibody or an antigen binding portion thereof. In one embodiment, the ligand or receptor comprises a His tag, C-myc tag (EQKLISEEDL), Flag tag (DYKDDDDK), SteptTag (WSHPQFEK), GST, thioredoxin, cellulose binding domain, chitin binding domain, metal binding domain, e.g., zinc binding domains or calcium binding domains such as those from calcium-binding proteins, e.g., calmodulin, or maltose binding protein. In one embodiment, the microbe is a Streptococcus, E. coli or Staphylococcus.

The invention also provides a method to detect antimicrobial activity in vitro, comprising: providing the support; introducing a culture of cells or a physiological sample to the support; and detecting whether the microbe in one or more of the defined areas is viable or has an altered property. In one embodiment, the sample is an airway surface liquid sample. In one embodiment, the altered property is relative to a corresponding microbe that is on a control support. In one embodiment, the altered property is relative to a corresponding microbe on the same support but in a different defined area.

### BRIEF DESCRIPTION OF THE FIGURES

FIG. 1. Bacterial killing is impaired in CF ASL. A) Schematic showing biotin/streptavidin linking S. aureus to gold grids that were placed on the airway surface. After removal, bacteria were exposed to fluorescent live/dead stain (SYTO 9/propidium iodide), imaged, and counted. B) Scanning electron photomicrographs of bacteria-coated grid (top), grid bar (middle), and individual bacteria (bottom). C) Image of bacteria-coated grid (green=live, red=dead) after placement for 5 min on tracheal surface of 1-month-old, wild-type pig. Bottom shows percentage of bacteria that were dead after immersion in saline, water, 70% ethanol, or placement on tracheal surface. n=3 each. Here and elsewhere, bars are SEM. D) S. aureus-coated grids were placed on tracheal surface of newborn CF and non-CF pigs for indicated times. Data are percentage dead bacteria. Each set of 3 time-points is from a single animal. Letters (a-f) indicate littermates; there is no 1 minute time point for CF pig in litter c due to experimental error. For each pig, 2-3 grids were used at each time point, 5-16 fields were counted per grid, each field contained about 100-1000 bacteria, and data from each field were averaged. Operators were blinded to genotype. CF was different from non-CF at all time points, P<0.01. E) S. aureus-coated grids were placed for 30 seconds on tracheal surface before and about 5 minutes after methacholine stimulated secretion. n=6/genotype. *P<0.02. For each genotype, data with and without methacholine do not differ significantly. F. ASL was removed from methacholine-stimulated pigs, bacteria (1×106 CFU/ml) were incubated in 1 μL ASL for 10 minutes in a micro CFU assay, and bacteria were counted by dilution plating. n=6/genotype. *P<0.05. G. S. aureus-coated grids were placed on surface of primary epithelial cultures for indicated times, and percentage dead bacteria was determined. Each set of data points represents mean from epithelia from a different animal. CF was different from non-CF at all time points, P<0.001. H. P. aeruginosa-coated grids were placed on surface of cultured epithelia for 5 min. n=5 cultures from different pigs per genotype. *P<0.001. I. S. aureus in 100 nl H2O were applied to apical surface of cultured epithelia. Apical surface was washed 24 hr later and bacteria counted by dilution plating. Data are % of epithelia that showed no bacterial growth. n=18/genotype. *P<0.005, Fisher's exact test.

FIG. 2. Transcript levels of lysozyme, lactoferrin, S100A9 and PBD-2 were measured with quantitative RT-PCR in trachea and bronchus tissue (n=6) and in primary cultures of tracheal epithelia (n=8) of non-CF and CF pigs. Data are average fold change (CF/non-CF)±SEM.

FIG. 3. CF and non-CF ASL have similar antimicrobial concentrations and aggregate antimicrobial activity under optimal conditions. A) Lysozyme concentration was measured with lysoplates; n=8/genotype. Quantitative western blots assayed lactoferrin, PLUNC, and SP-A; data are relative intensity of blots, n=12/genotype. B) 60 μL isotonic xylitol was added to apical surface of airway cultures (diluting ASL about 100:1). 3 minutes later, two S. aureus-coated grids were placed on epithelial surface for 1 minute, then removed and counted. n=6 epithelia/genotype, each from different pigs. C) ASL was removed from cultured epithelia by washing with 100 μL H2O. ASL was incubated with S. aureus (3.3×103 CFU/ml) for 60 minutes, and micro CFU assays were used to measure antimicrobial activity. n=12/genotype. D) Methacholine-stimulated ASL was diluted 1:100 in H2O, incubated with S. aureus (1×106 CFU/ml) for 60 minutes, and CFU assays were used to measure antimicrobial activity. n=5/genotype. E) Radial diffusion assays were used to measure antimicrobial activity of ASL collected from epithelial cultures. n=6/genotype. FIG. 4. The apical surface of primary cultures of non-CF and CF airway epithelia was rinsed with 70 μl xylitol (isosmotic with basolateral culture media). Serial dilutions (as shown) were inoculated with 8.5×10³CFU/ml S. aureus (SA43) and incubated for 1 hour at 37° C., followed by dilution plating to quantify remaining bacteria. n=6 pigs, 2 replicate cultures per pig were averaged. p>0.05 for all dilutions.

FIG. 5. ASL pH is more acidic in CF than non-CF. A. ASL was collected under basal conditions from tracheal surface using Parafilm-coated paper; Na+ and K+ concentrations were measured as described in Methods (Table 3). n=8 non-CF and 6 CF. B. ASL Na+ and K+ concentrations in methacholine-stimulated ASL. n=16 non-CF and 14 CF pigs. *P<0.05. C. ASL pH measured in vivo using pH-sensitive planar optical probe placed on tracheal surface. n=6 non-CF and 7 CF; littermates were used with one extra CF. *P<0.05. Studies were done with animals in 5% CO2; therefore, ASL CO2 concentration was likely>5% due to CO2 production by the pigs. D. Methacholine-stimulated ASL was removed, and pH was measured with a micro-optical pH probe. pH was measured 10 minutes after removal in ambient CO2, which likely increased pH values compared to in vivo. n=10 pigs/genotype; littermates were used. *P<0.0005. E. ASL pH measured in cultured airway epithelia using fluorescent pH indicator. N=5epithelia/genotype, each from a different pig. *P<0.01. Calculated HCO3-concentrations using measured pH, the 5% CO2 concentration, and Henderson-Hasselbalch equation were non-CF 28.1±4.2 mM (n=8) and CF 13.1±2.4 mM (n=8), p=0.007.

FIG. 6. Increasing ASL pH enhances antimicrobial activity. A) Methacholine-stimulated ASL was removed from non-CF pigs, pH was adjusted with HCl, and antimicrobial activity was measured with S. aureus-coated grids. n=6. B) Effect of pH on antimicrobial activity of 1.25 mg/ml lysozyme and 4 mg/ml lactoferrin in S. aureus luminescence assay. Data are relative luminescence compared to control at 30 minutes for lysozyme and 60 minutes for lactoferrin. Similar results were obtained with E. coli. C) Tracheas of non-CF pigs were exposed to 0% or 15% CO2 in vivo. pH was measured with pH-sensitive planar electrode. n=6/genotype. Bacterial killing was measured with S. aureus-coated grids placed on surface for 30 seconds n=4/genotype. *P<0.05. D) NaHC03 or NaCl (50 μL, 100 mM) was aerosolized onto airway surface of CF pigs. pH (n=6/genotype) and bacterial killing (n=5/genotype) were measured as in 4c. *P<0.05.

FIG. 7. As a control for the data in FIG. 6A, bacteria-coated grids were immersed in a solution containing 100 mM HEPES adjusted to the indicated pH. Each data point represents average data from an individual grid; two grids were tested for each pH value.

FIG. 8. Effect of pH on antimicrobial activity of lysozyme and lactoferrin in an E. coli luminescence assay. Data are the concentrations of lysozyme and lactoferrin required to inhibit E. coli luminescence by 50% (IC50).

FIG. 9. As a control for the data in FIG. 6B, we tested the effect of pH on viability of S. aureus after incubation for 30 minutes in 1% TSB (left panel) or 60 minutes in pure water (right panel). Both solutions contained 10 mM HEPES adjusted to the indicated pH. Data are relative luminescence compared to baseline (time 0) (left panel), or CFU relative to baseline (right panel).

FIG. 10. Average number of bacteria per microscopic field. Data are from the bacteria-coated grids used for the in vivo and in vitro experiments shown in FIGS. 1d and 1g.

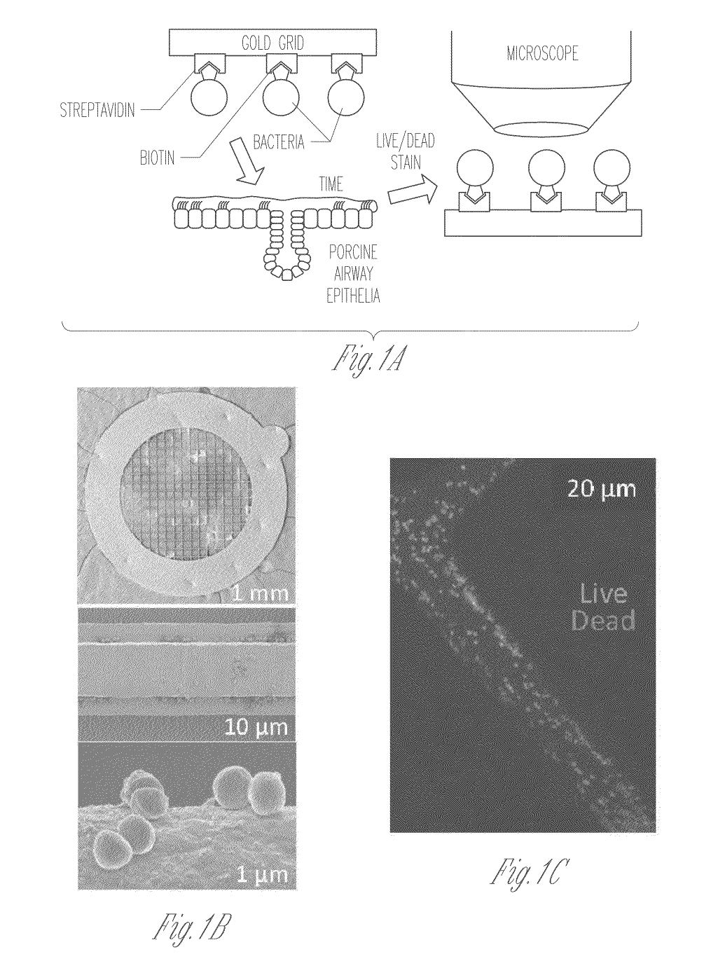

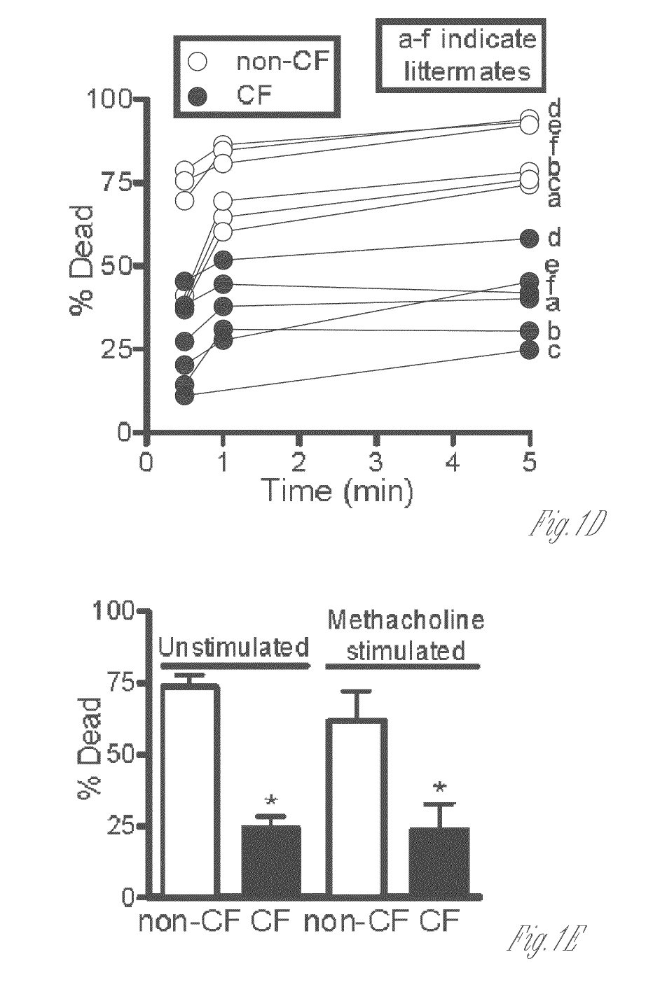

FIG. 1I. Comparison of bacterial viability assessed by CFU and BacLight live/dead staining. S. aureus (SA43) were grown to late log phase in TSB, and live bacteria were mixed with dead bacteria (incubated with 70% alcohol at room temperature for 1 hour) in fixed ratios as indicated

### DETAILED DESCRIPTION

Cystic fibrosis (CF) is a life-shortening disease caused by mutations in the cystic fibrosis transmembrane conductance regulator (CFTR) gene¹. Although bacterial lung infection and the resulting inflammation cause most of the morbidity and mortality, how loss of CFTR first disrupts airway host defense has remained uncertain²⁻⁶. What abnormalities impair eradication when a bacterium lands on the pristine surface of a newborn CF airway? To investigate these defects, the viability of individual bacteria immobilized on solid grids and placed on the airway surface was interrogated. As a model, CF pigs were studied, which spontaneously develop hallmark features of CF lung disease⁷⁻⁸. At birth, their lungs lack infection and inflammation, but have a reduced ability to eradicate bacteria⁸. Herein it is shown that in newborn wild-type pigs, the thin layer of airway surface liquid (ASL) rapidly killed bacteria in vivo, when removed from the lung, and in primary epithelial cultures. Lack of CFTR reduced bacterial killing. ASL pH was found to be more acidic in CF, and reducing pH inhibited the antimicrobial activity of ASL. Reducing ASL pH diminished bacterial killing in wild-type pigs, and increasing ASL pH rescued killing in CF pigs. These results directly link the initial host defense defect to loss of CFTR, an anion channel that facilitates HCO₃⁻ transport⁸⁻¹³. Without CFTR, airway epithelial HCO₃⁻ secretion is defective, ASL pH falls and inhibits antimicrobial function, and thereby impairs killing of bacteria that enter the newborn lung. These findings suggest that increasing ASL pH might prevent the initial infection in patients with CF and that assaying bacterial killing could report on the benefit of therapeutic interventions.

Proposed origins of CF lung disease include reduced mucociliary clearance due to decreased ASL volume or altered mucus, reduced bacterial killing by ASL antimicrobials, defective bacterial elimination by phagocytes, abnormal inflammatory responses, reduced or increased bacterial binding by airway epithelia, and other defects²⁻⁶. One or more of these defects could be responsible. Two factors have made it difficult to distinguish between hypotheses and identify initiating insults. First, as with many diseases, clinical manifestations may not reflect original defects, but it is problematic to study CF at its onset in newborn human infants. Second, mice with disrupted CFTR genes do not develop typical CF¹⁴.

To circumvent these obstacles, CFTR^{-/-} pigs (CF pigs) were generated⁷. Within months of birth, CF pigs spontaneously develop airway inflammation, infection, tissue remodeling, mucus accumulation, and airway obstruction⁷⁻⁸, hallmark features of CF lungs. Although at birth they exhibit none of these features, they already manifest a host defense defect against bacteria. Thus, newborn CF pigs provide an unprecedented opportunity to investigate mechanisms impairing host defense and initiating disease because they allow CF:non-CF comparisons without secondary confounds.

In previous work, Staphylococcus aureus was instilled into airways and four hours later found more bacteria in CF than non-CF pigs⁸. However, that study revealed little about responsible mechanisms; it was not known whether bacteria were removed or killed within the lung, whether bacteria grew following instillation, whether phagocytic cells eliminated bacteria, whether bacteria bound to surfaces, or whether deposition and sampling were identical in all animals.

To investigate initial host defense defects, a simple assay was developed that tested viability of individual bacteria. Biotin was chemically linked to S. aureus, streptavidin was bound to gold grids, and those were combined to attach S. aureus to grids (FIG. 1A,B). S. aureus was selected because it is frequently isolated from porcine CF lungs, and it is the most common organism isolated from young children with CF^{8,15}. A fluorescent live/dead stain revealed the state of bacteria. Exposing grids to ethanol killed most S. aureus (FIG. 1C). Importantly, placing grids on the porcine tracheal surface in vivo also killed bacteria.

In 6-15 hour-old pigs, a small tracheal incision was made and bacteria-coated grids were placed on the airway surface. Even 30-sec applications on non-CF airways killed S. aureus (FIG. 1D). Applying grids to littermate CF pigs killed approximately half as many bacteria. Methacholine was administered to stimulate secretion of submucosal glands, which produce substantial amounts of antimicrobials¹⁶⁻¹⁷, and to allow for the collection of ASL for other studies. After methacholine, CF:non-CF differences persisted (FIG. 1E). It was predicted that antimicrobial activity would also be detected if methacholine-stimulated ASL was removed and studied with conventional colony-forming unit (CFU) assays. Indeed, bacterial killing was reduced in CF secretions (FIG. 1F).

S. aureus-coated grids were also applied to primary cultures of porcine airway epithelia and found reduced killing in CF (FIG. 1G). Previous data suggest that the host-defense defect involves many different bacteria^{8,15}. Therefore, Pseudomonas aeruginosa-coated grids were tested and found that there was defective killing by CF epithelia (FIG. 1H). S. aureus coated was added directly to cultured epithelia. Most non-CF epithelia eliminated low inocula of bacteria, but bacteria grew on most CF epithelia (FIG. 1I). At the highest inocula, S. aureus infected both CF and non-CF epithelia.

These data indicate that ASL rapidly kills bacteria, and CF impairs killing. The defect was partial, as CF ASL retained some activity. The assays allow several conclusions, a) Defective bacterial killing was not due to dysfunctional mucociliary clearance or abnormal killing by phagocytes; neither would explain results with grids in vivo or studies of cultured epithelia. b) the CF:non-CF differences cannot be attributed to altered bacterial-epithelial binding because of the difference with bacteria attached to grids and with ASL studied ex vivo. c) The earlier finding that newborn CF airways lacked inflammation⁸ and the killing defect in cultured epithelia indicate that abnormal inflammation was not responsible. d) The bacteria-coated grid method also excludes differences in bacterial delivery, sampling, or growth. Therefore, we reasoned that defective killing arose either from reduced amounts of ASL antimicrobial factors or inhibition of their function.

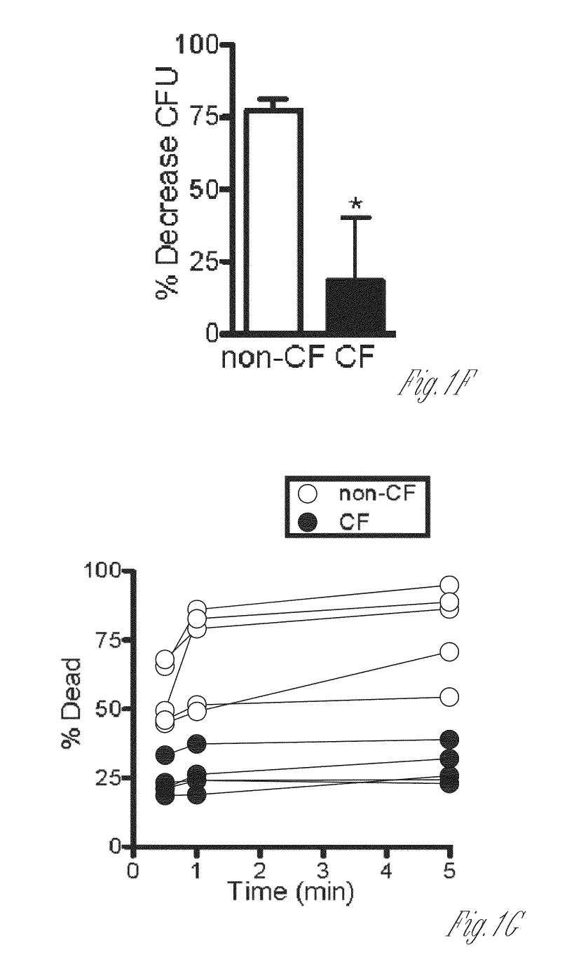

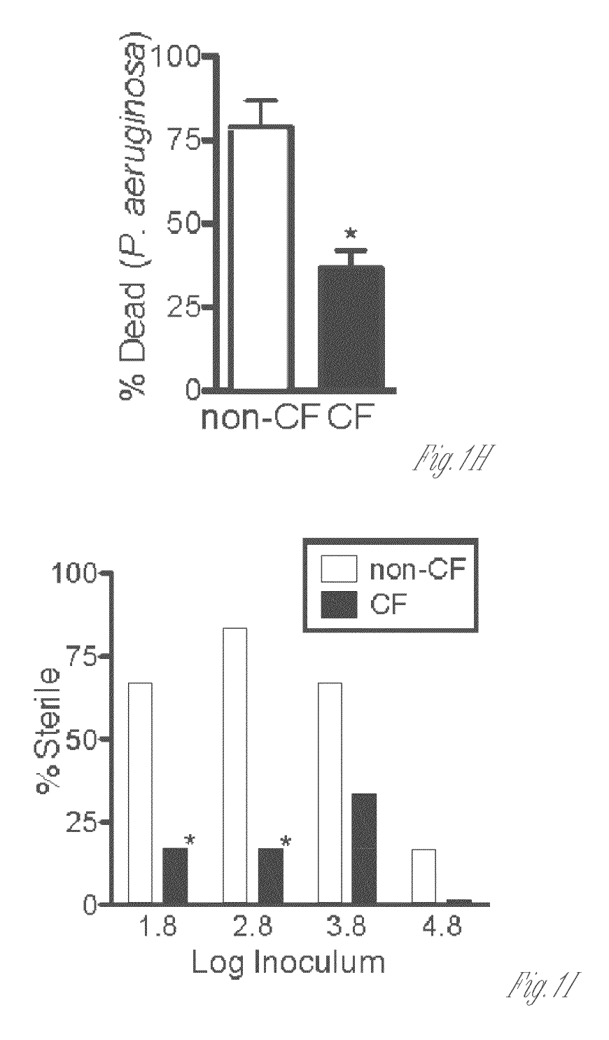

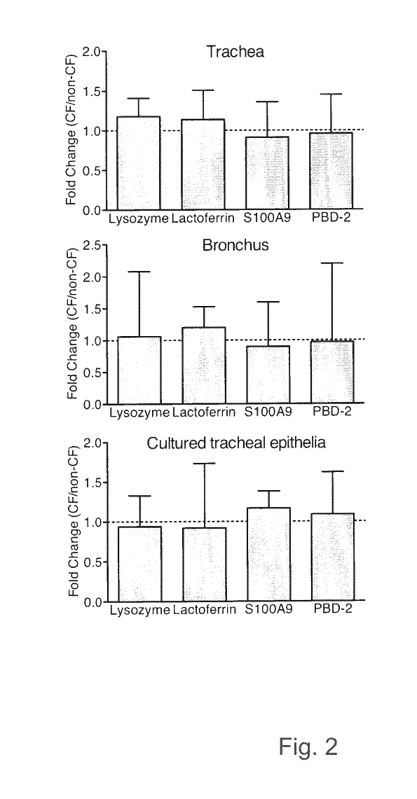

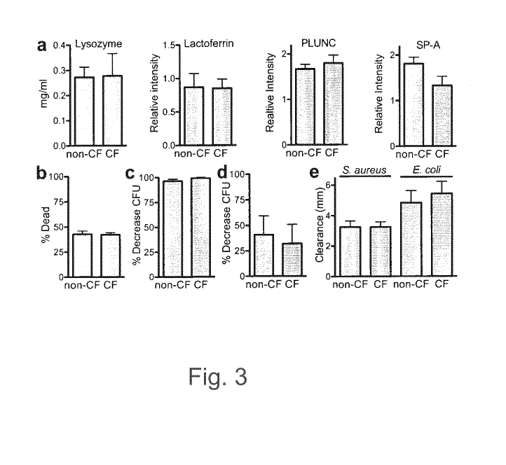

Antimicrobials were investigated by measuring mRNA, protein, and aggregate activity under optimal conditions. The abundance of transcripts for secreted antimicrobial proteins (Table 1, FIG. 2) and proteins with known host defense functions revealed no consistent differences between genotypes (Table 2). In methacholine-stimulated ASL, concentrations of the two most abundant antimicrobials, lysozyme and lactoferrin, as well as PLUNC and SP-A did not differ by genotype (FIG. 3A). To assay aggregate ASL antimicrobial function, we did four experiments in which we maximized activity by reducing ionic strength close to zero ^{16,18,19}. First, isotonic, salt-free buffer was added to apical surfaces of cultured airway epithelia. Under these controlled conditions, both genotypes showed equivalent killing of bacteria on grids (FIG. 3B). Second, ASL removed from cultured CF and non-CF epithelia with water killed bacteria to the same extent (FIG. 3C, FIG. 4). Third, ASL removed from pigs and diluted 1:100 with water showed genotype-independent killing (FIG. 3D). Fourth, radial diffusion assays with 10 mM Na phosphate in 1% agarose revealed areas of clearance for S. aureus and E. coli that were similar for both genotypes (FIG. 3E). These data indicate that non-CF and CF ASL had similar amounts of antimicrobials. Thus, they suggested that CF:non-CF bacterial killing disparities derived from other differences in ASL composition.

TABLE 1
Abundance of mRNAs for secreted antimicrobial proteins in CF and non-CF samples.
Data are from porcine trachea (n = 5 non-CF and 7 CF), porcine bronchus
(n = 7/genotype), and primary cultures of airway epithelia (n = 6 non-CF and 5 CF). Gene
expression levels of all annotated mRNAs with identified direct antimicrobial activity were
assessed with Affymetrix Porcine GeneChips. “Fold Change” represents CF vs. non-CF
RMA normalized signal intensities; negative fold change indicates decreased expression in
CF. FDR indicates false discovery rate; values < 0.1 were considered significant. Of the 13
mRNAs listed, none showed a statistically significant difference between CF and non-CF.

| | | | | | Cultured Tracheal | |
|---|---|---|---|---|---|---|
| | Trachea | | Bronchus | | Epithelia | |
| Antimicrobial | Fold | | Fold | | Fold | |
| Proteins | Change | FDR | Change | FDR | Change | FDR |
| BPI | −1.02 | 0.68 | 1.04 | 0.64 | −1.02 | 0.86 |
| Cathelicidin | 1.26 | 0.57 | 1.24 | 0.55 | 1.04 | 0.85 |
| Lactoferrin | 2.06 | 0.58 | 1.93 | 0.60 | −1.53 | 0.58 |
| Lysozyme | 1.04 | 0.64 | 1.01 | 0.80 | −1.48 | 0.86 |
| PBD-1 | −1.01 | 0.72 | 1.02 | 0.72 | 1.08 | 0.84 |
| PBD-2 | −1.34 | 0.56 | 1.04 | 0.79 | 1.88 | 0.55 |
| PBD-103* | −1.02 | 0.69 | 1.01 | 0.78 | 1.15 | 0.38 |
| PBD-111* | 1.02 | 0.65 | −1.00 | 0.79 | 1.07 | 0.81 |
| PBD-125* | 1.01 | 0.74 | 1.03 | 0.64 | 1.05 | 0.80 |
| PBD-129* | −1.00 | 0.78 | −1.01 | 0.79 | −1.04 | 0.74 |
| PLUNC | −1.36 | 0.61 | −1.61 | 0.62 | 1.04 | 0.76 |
| S100A8 | 1.32 | 0.56 | 1.15 | 0.57 | −1.33 | 0.80 |
| S100A9 | 5.12 | 0.56 | 1.91 | 0.68 | −1.50 | 0.81 |

PBD = Porcine Beta-Defensin.
*Indicates porcine ortholog of the indicated human defensin¹⁷.

TABLE 2
Abundance of host defense mRNAs in CF and non-CF samples.
Gene expression levels are those of all annotated mRNAs with identified host defense
properties. Data and analysis are as described in legend of Table 1. Of the 87 mRNAs
listed, none showed a statistically significant difference between CF and non-CF.

| | | | | | Cultured Tracheal | |
|---|---|---|---|---|---|---|
| | Trachea | | Bronchus | | Epithelia | |
| Host | Fold | | Fold | | Fold | |
| defense | Change | FDR | Change | FDR | Change | FDR |
| A2M | 1.36 | 0.56 | 1.11 | 0.74 | 1.01 | 0.85 |
| AMBP | 1.01 | 0.72 | 1.01 | 0.74 | 1.07 | 0.85 |
| B2M | −1.07 | 0.62 | −1.09 | 0.66 | 1.05 | 0.72 |
| C1QA | −1.08 | 0.70 | 1.01 | 0.81 | 1.16 | 0.71 |
| C1QB | −1.16 | 0.63 | −1.08 | 0.79 | 1.27 | 0.72 |
| C1QC | −1.01 | 0.78 | −1.10 | 0.79 | 1.04 | 0.79 |
| C1R | 1.13 | 0.66 | −1.40 | 0.51 | −1.08 | 0.87 |
| C1S | 1.02 | 0.78 | −1.42 | 0.52 | −1.32 | 0.66 |
| C2 | −1.01 | 0.76 | −1.02 | 0.77 | 1.05 | 0.82 |
| C3 | 1.23 | 0.59 | 1.13 | 0.64 | −1.22 | 0.75 |
| C4B | −1.09 | 0.70 | −1.06 | 0.75 | 1.26 | 0.77 |
| C4BPA | −1.03 | 0.76 | −1.20 | 0.69 | 1.03 | 0.82 |
| C4BPB | −1.09 | 0.72 | −1.21 | 0.51 | 1.06 | 0.87 |
| C5 | 3.95 | 0.56 | 1.53 | 0.76 | −1.24 | 0.61 |
| C6 | 1.01 | 0.76 | 1.13 | 0.60 | −1.02 | 0.85 |
| C7 | 1.56 | 0.62 | 1.14 | 0.79 | 1.01 | 0.87 |
| C8A | −1.01 | 0.74 | 1.03 | 0.68 | 1.23 | 0.36 |
| C8B | −1.02 | 0.64 | 1.02 | 0.54 | 1.08 | 0.69 |
| C8G | 1.03 | 0.66 | 1.03 | 0.67 | 1.10 | 0.53 |
| C9 | −1.01 | 0.78 | −1.03 | 0.76 | −1.31 | 0.73 |
| CCL2 | 1.60 | 0.56 | −2.62 | 0.47 | 1.02 | 0.81 |
| CCL8 | −1.01 | 0.74 | −1.27 | 0.50 | 1.27 | 0.63 |
| CFB | 1.12 | 0.68 | 1.43 | 0.65 | 1.19 | 0.53 |
| CFD | −1.06 | 0.65 | −1.01 | 0.81 | 1.06 | 0.83 |
| CFH | −1.08 | 0.73 | −1.01 | 0.81 | −1.05 | 0.76 |
| CFI | −1.56 | 0.56 | −1.65 | 0.59 | −1.07 | 0.77 |
| CFP | −1.13 | 0.56 | −1.00 | 0.81 | 1.30 | 0.71 |
| CRP | 1.01 | 0.76 | −1.02 | 0.68 | 1.02 | 0.75 |
| CST11 | −1.01 | 0.74 | 1.02 | 0.71 | −1.02 | 0.86 |
| CST3 | −1.00 | 0.78 | −1.03 | 0.80 | 1.77 | 0.11 |
| CST6 | −1.05 | 0.69 | −1.19 | 0.59 | 1.04 | 0.86 |
| CST9L | −1.03 | 0.56 | −1.03 | 0.67 | 1.02 | 0.82 |
| CSTA | 1.09 | 0.56 | 1.05 | 0.69 | 1.02 | 0.82 |
| CSTB | 1.11 | 0.59 | −1.17 | 0.55 | 1.04 | 0.60 |
| CTSA | −1.00 | 0.78 | 1.06 | 0.65 | 1.13 | 0.83 |
| CTSB | 1.16 | 0.56 | −1.04 | 0.77 | 1.11 | 0.66 |
| CTSC | 1.21 | 0.62 | 1.17 | 0.70 | 1.04 | 0.87 |
| CTSD | 1.10 | 0.60 | 1.01 | 0.81 | 1.10 | 0.68 |
| CTSF | −1.23 | 0.61 | −1.06 | 0.79 | 1.06 | 0.72 |
| CTSG | −1.07 | 0.56 | −1.01 | 0.75 | 1.06 | 0.87 |
| CTSH | 1.41 | 0.56 | 1.04 | 0.80 | 1.12 | 0.83 |
| CTSK | 1.11 | 0.66 | −1.15 | 0.71 | −1.10 | 0.79 |
| CTSL1 | 1.03 | 0.73 | −1.15 | 0.62 | 1.22 | 0.67 |
| CTSL2 | 1.05 | 0.64 | −1.01 | 0.80 | −1.07 | 0.84 |
| CTSO | −1.01 | 0.71 | 1.00 | 0.81 | 1.02 | 0.77 |
| CTSS | −1.03 | 0.75 | −1.20 | 0.72 | −1.08 | 0.78 |
| CTSZ | 1.70 | 0.56 | 1.27 | 0.71 | 1.09 | 0.82 |
| Elafin | 1.04 | 0.57 | −1.02 | 0.73 | −1.12 | 0.78 |
| Elafin | −1.12 | 0.77 | −1.29 | 0.62 | 1.40 | 0.60 |
| FCN2 | −1.37 | 0.65 | −1.37 | 0.65 | 1.13 | 0.71 |
| IL12A | −1.02 | 0.57 | −1.03 | 0.55 | −1.07 | 0.79 |
| IL17B | 1.00 | 0.77 | 1.04 | 0.63 | 1.12 | 0.78 |
| IL18 | 1.10 | 0.60 | −1.29 | 0.45 | −1.15 | 0.59 |
| IL1A | −1.01 | 0.75 | −1.01 | 0.80 | −1.33 | 0.53 |
| IL1RN | 1.17 | 0.59 | 1.19 | 0.55 | 1.13 | 0.73 |
| IL2 | 1.03 | 0.56 | 1.01 | 0.72 | 1.03 | 0.84 |
| IL33 | 1.11 | 0.59 | −1.19 | 0.49 | −1.05 | 0.53 |
| IL4 | −1.01 | 0.70 | −1.02 | 0.58 | 1.00 | 0.85 |
| LBP | 1.28 | 0.64 | −1.06 | 0.62 | −3.70 | 0.11 |
| LCN1L1 | −3.76 | 0.56 | −3.51 | 0.57 | 1.09 | 0.80 |
| LCN6 | −1.01 | 0.70 | −1.01 | 0.77 | −1.08 | 0.66 |
| LPO | −1.08 | 0.57 | 1.15 | 0.71 | 1.06 | 0.79 |
| MASP1 | −1.07 | 0.59 | −1.01 | 0.75 | 1.07 | 0.76 |
| MASP2 | −1.15 | 0.65 | 1.20 | 0.62 | −1.14 | 0.47 |
| MBL2 | 1.00 | 0.77 | −1.01 | 0.76 | 1.07 | 0.85 |
| SERPINA1 | −1.16 | 0.56 | 1.00 | 0.81 | 1.01 | 0.81 |
| SERPINA11 | 1.02 | 0.65 | −1.01 | 0.79 | 1.15 | 0.41 |
| SERPINA3 | 1.01 | 0.72 | 1.00 | 0.80 | −1.25 | 0.80 |
| SERPINA5 | −1.04 | 0.58 | 1.04 | 0.54 | −1.04 | 0.79 |
| SERPINA7 | −1.09 | 0.64 | −1.05 | 0.72 | 2.14 | 0.76 |
| SERPINB1 | −1.00 | 0.77 | 1.01 | 0.69 | 1.13 | 0.75 |
| SERPINB2 | 1.28 | 0.56 | −1.06 | 0.59 | −1.14 | 0.78 |
| SERPINB5 | −1.27 | 0.66 | −1.63 | 0.42 | −1.23 | 0.82 |
| SERPINB7 | −1.01 | 0.69 | −1.01 | 0.71 | −1.51 | 0.78 |
| SERPINC1 | 1.06 | 0.71 | 1.05 | 0.74 | −1.01 | 0.82 |
| SERPIND1 | 1.01 | 0.74 | −1.03 | 0.62 | 1.06 | 0.82 |
| SERPINE1 | 2.77 | 0.56 | 1.94 | 0.53 | −1.11 | 0.63 |
| SERPINE2 | −1.04 | 0.56 | 1.01 | 0.69 | 1.05 | 0.86 |
| SERPINF1 | −1.30 | 0.59 | −1.79 | 0.44 | −1.19 | 0.78 |
| SERPINF2 | −1.02 | 0.74 | −1.00 | 0.80 | 1.14 | 0.77 |
| SERPING1 | 1.19 | 0.56 | −1.15 | 0.69 | −1.02 | 0.83 |
| SERPINI1 | 1.01 | 0.73 | 1.04 | 0.75 | −1.11 | 0.72 |
| SFTPA1B | 3.23 | 0.56 | 1.80 | 0.71 | −1.06 | 0.84 |
| SFTPD | 3.21 | 0.56 | 1.74 | 0.70 | −1.28 | 0.54 |
| TGFB1 | 1.35 | 0.56 | 1.09 | 0.76 | 1.05 | 0.85 |
| TGFB2 | 1.21 | 0.56 | 1.07 | 0.77 | −1.02 | 0.86 |
| VSIG4 | -1.05 | 0.70 | 1.12 | 0.69 | −1.15 | 0.64 |

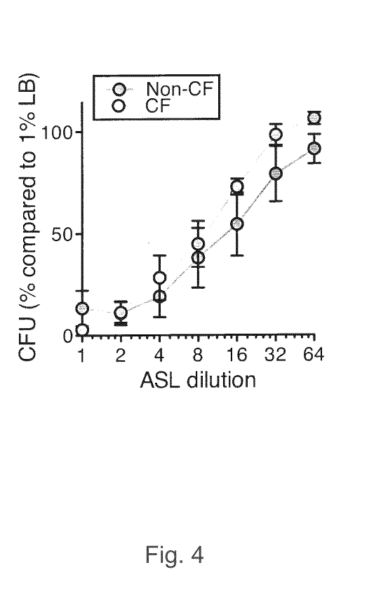

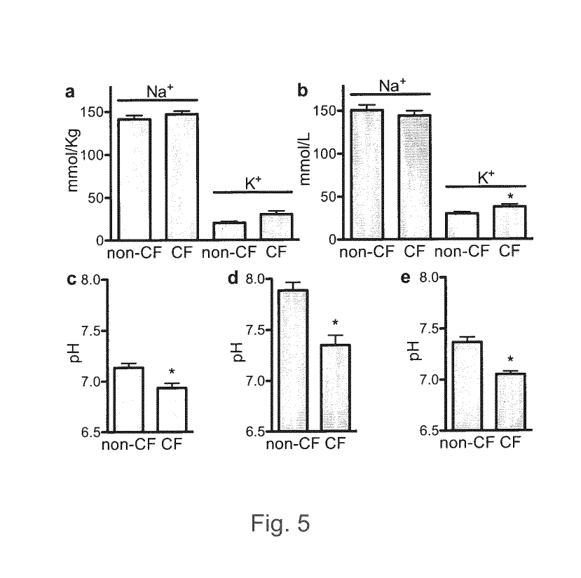

An increased ionic strength inhibits activity of many antimicrobials^{16,18,19}. Studies of human CF airway epithelia in culture or xenografts reported either higher^{20,21} or the same^{22,23} ASL NaCl concentrations as non-CF, but an in vivo study²⁴ reported similar concentrations. Therefore, Na⁺ and K⁺ concentrations were measured in ASL collected from newborn pigs and it was found that they did not differ by genotype (FIG. 5A). In addition, ASL collected after methacholine stimulation showed ion concentrations similar to those measured under basal conditions and only minor differences in K⁺ concentration between CF and non-CF (FIG. 5B). Thus, different ASL Na⁺ and K⁺ concentrations do not explain defective bacterial killing in CF.

Earlier studies indicated that pH can affect antimicrobial activity^{19,25}. Human and porcine airway epithelia exhibit CFTR-dependent HCO₃⁻ secretion^{11,12}, and CF reduces ASL pH^{26,27}. To assess ASL pH in vivo, a planar pH-sensitive probe was placed on the tracheal surface. pH was lower in CF than non-CF ASL (FIG. 5C). Methacholine-stimulated ASL removed from CF pigs and measured with an optical pH probe was more acidic than non-CF (FIG. 5D); pH was measured after removal in ambient CO₂, likely contributing to the higher absolute pH values. ASL pH was also measured in primary airway epithelial cultures using a fluorescent pH indicator and found reduced pH in CF (FIG. 5E). Although absolute pH values varied in different preparations, in all three, CF ASL was more acidic.

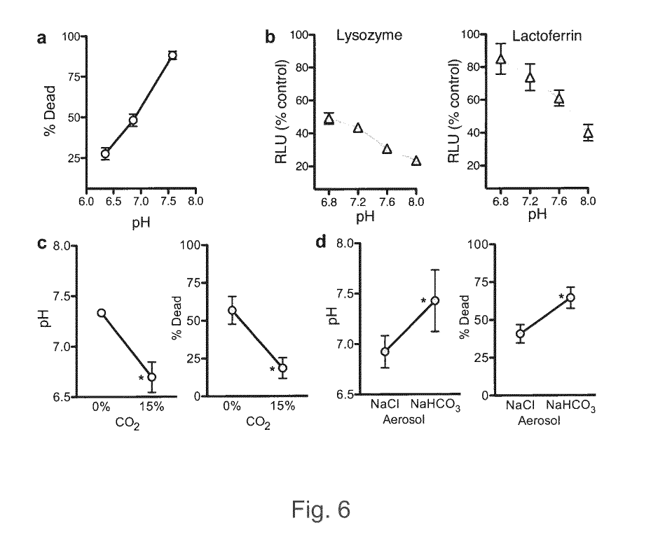

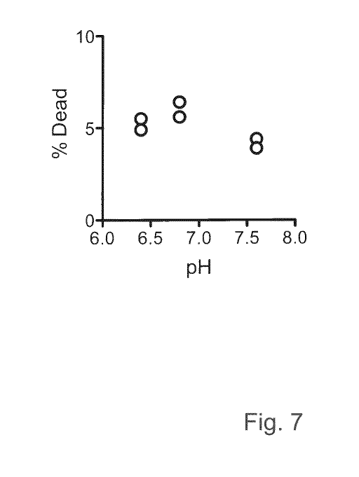

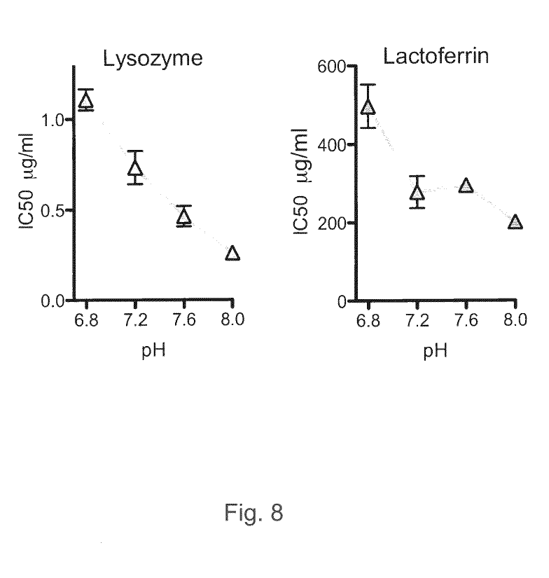

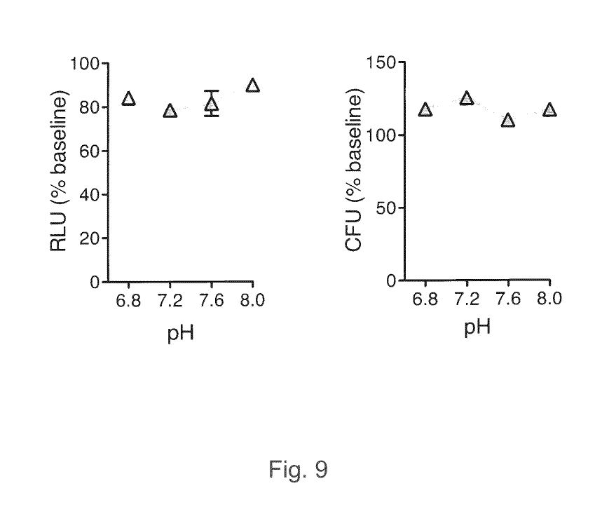

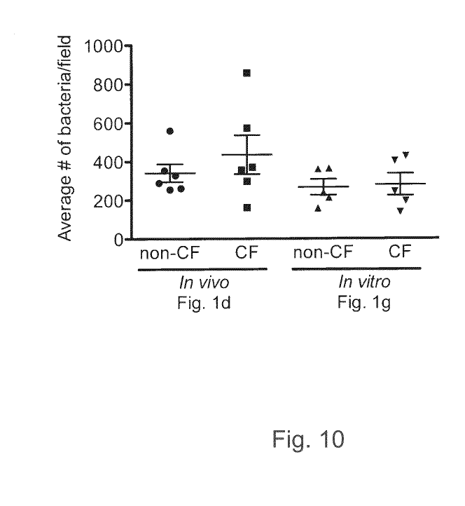

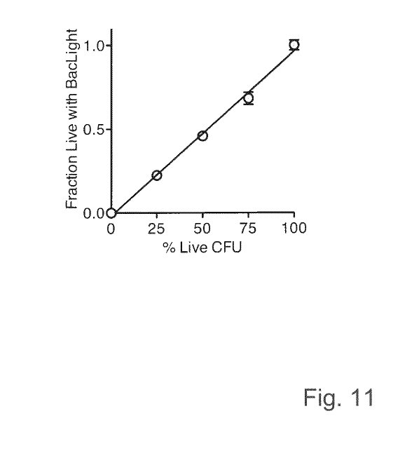

It was tested whether pH affects ASL antimicrobial activity by removing ASL from newborn non-CF pigs, adjusting pH, and applying S. aureus-coated grids. Killing was pH-dependent, increasing as pH increased (FIG. 6A, FIG. 7). Lysozyme and lactoferrin were also tested; increasing pH increased S. aureus and E. coli killing (FIG. 6B, FIG. 8, FIG. 9).

If pH is responsible for differences in bacterial killing, it was predicted that reducing ASL pH would inhibit bacterial killing in wild-type pigs, and raising pH would enhance killing in CF pigs. In non-CF pigs, elevating airway CO₂ reduced ASL pH and inhibited bacterial killing (FIG. 6C). In CF pigs, NaHCO₃ was aerosolized into the trachea. Compared to NaCl, NaHCO₃ increased ASL pH and enhanced killing (FIG. 6D).

TABLE 3
Numerical data for FIG. 5 and 6. Data are mean ± SEM (n).

| FIG. 5a | Non-CF | CF | |
|---|---|---|---|
| Na⁺ | 141.4 ± 4.4 (8) | 147.1 ± 3.7 (6) | |
| K⁺ | 20.1 ± 1.8 (8) | 29.9 ± 4.1 (6) | |
| FIG. 5b | Non-CF | CF | |
| Na⁺ | 150.5 ± 6.2 (16) | 144.0 ± 5.6 (14) | |
| K⁺ | 29.9 ± 2.1 (16) | 37.9 ± 2.9 (14) | |
| FIG. 5c | Non-CF | CF | |
| pH | 7.14 ± 0.04 (6) | 6.94 ± 0.05 (7) | |
| FIG. 5d | Non-CF | CF | |
| pH | 7.89 ± 0.08 (10) | 7.35 ± 0.10 (10) | |
| FIG. 5e | Non-CF | CF | |
| pH | 7.37 ± 0.05 (5) | 7.5 ± 0.03 (5) | |
| FIG. 6a | pH 6.35 | pH 6.86 | pH 7.58 |
| % dead | 27.5 ± 3.7 (8) | 48.1 ± 3.8 (n = 16) | 88.1 ± 2.5 (n = 16) |
| FIG. 6b | Lysozyme | Lactoferrin | |
| pH 6.8 | 49.1 ± 3.4 | 85.0 ± 9.3 | |
| pH 7.2 | 43.5 ± 1.5 | 73.6 ± 8.2 | |
| pH 7.6 | 30.6 ± 2.3 | 60.8 ± 4.8 | |
| pH 8.0 | 23.4 ± 0.8 | 39.8 ± 5.0 | |
| FIG. 6c | 0% CO₂ | 15% CO₂ | |
| pH | 7.33 ± 0.03 (7) | 6.89 ± 0.15 (6) | |
| % dead | 56.8 ± 9.1 (4) | 18.5 ± 6.8 (4) | |
| FIG. 6d | NaCl | NaHCO₃ | |
| pH | 6.92 ± 0.16 (6) | 7.43 ± 0.30 (7) | |
| % dead | 40.8 ± 6.1 (5) | 64.4 ± 7.0 (5) | |

These results directly link CFTR mutations to defective bacterial eradication. CFTR is an anion channel that conducts HCO₃⁻ and works in concert with C1⁻/HCO₃⁻ exchange and H⁺ secretion to regulate ASL pH ^{2,9,10,13}; its loss prevents airway epithelia from secreting HCO₃^{-11,12}. The resulting decreased ASL pH inhibits antimicrobial function, thereby impairing killing of bacteria that enter the lung. The present findings with bacteria-coated grids in vivo, ASL removed from pigs, and primary epithelial cultures all point to this defect.

What about other defects that might commence CF lung disease? First, progression from the pristine lung of a newborn to the chronically infected, inflamed, remodeled, and obstructed lung of a person with advanced CF entails many steps. The present findings suggest that reduced antibacterial activity may initiate this downward spiral. However additional abnormalities at the onset of disease cannot be excluded. These might include abnormal mucociliary clearance, bacterial binding, inflammation, or phagocytic function. As disease progresses, the relative importance of various defects may also change. As one example, reduced ASL pH might alter mucus secretion ² and thereby impair mucociliary clearance either at the genesis of disease or as mucus secretion increases with inflammation and remodeling. Second, ASL contains a complex mixture of peptides, proteins and lipids with individual^{16,28,29} and synergistic antimicrobial effects^{29,30}. The present data do not assess relative importance or abundance of each factor or CF:non-CF differences.

Several potential fates await bacteria that land on the airway surface. They might be killed. They might remain metabolically active, but unable to reproduce. They might replicate. And/or they might be removed. The balance between these options and their timing determines whether or not infection ensues. The grid assay disclosed herein tested one of these alternatives independent of the others, and the data indicate that ASL killed many, but not all bacteria. The speed of killing was remarkable, although killing might have continued after grids were withdrawn from ASL, rinsed and placed in indicator solution. Quick action is consistent with rapid bacterial permeabilization by antimicrobial proteins¹⁹. The other assays showing reduced numbers of growing bacteria (CFUs) after being placed on epithelial cultures or in ASL reflect consequences of immediate killing plus potential effects of ASL on replication. Results from earlier delivery of bacteria into porcine airways reflect all these host defenses plus bacterial removal and phagocytosis⁸.

Although multiple processes protect lungs when bacteria enter, ASL antimicrobials may be key to rapidly reducing numbers of viable organisms and thereby decreasing the probability that they will replicate, escape other mechanisms, and colonize the lung.

Aerosolizing HCO₃⁻ onto CF airways in vivo increased bacterial killing. Mechanistic findings and this result suggest that correcting ASL pH might prevent the initial CF infection. That might be accomplished by delivering HCO₃⁻ into airways, altering pH regulation by airway epithelia, enhancing activity of ASL antimicrobials, delivering pH-insensitive antimicrobials, or targeting mutant CFTR. These results also suggest that adapting the bacteria-coated grid method to assay bacterial killing in patients could be useful for assessing potential therapies. We also speculate that increasing ASL pH might prevent or treat airway infections in other diseases.

### Methods

CFTR^{-/-} and CFTR^{+/+} pigs

The generation of CFTR^{-/-} pigs has been reported^{1,2}. Animals were produced by mating CFTR male and female pigs. Newborn littermates were obtained from Exemplar Genetics. Animals were studied 8-15 hours after birth. Euthanasia was with IV Euthasol (Virbac). The University of Iowa Animal Care and Use Committee approved all animal studies.

### Preparation of Differentiated Primary Cultures of Airway Epithelia

Epithelial cells were isolated from the trachea and bronchi by enzymatic digestion, seeded onto collagen-coated, semi-permeable membranes (0.6 cm² Millicell-PCF; Millipore, Bedford, Mass.), and grown at the air-liquid interface as previously described^{3,4}. Culture medium, a 1:1 mixture of Dulbecco's modified Eagle's medium and Ham's FI2 medium (DMEM/F12), was supplemented with 2% Ultroser G (PALL France SAS; Saint Germain-en-Laye, France). Differentiated epithelia were used at least 14 days after seeding.

### Bacteria-Coated Grids

Gold grids (TEM grids, 200 mesh, Ted Pella Inc.) were immersed in 1 mM 11-mercaptoundecanoic acid (HS(CH₂)₁₀COOH, MUA, Aldrich) solution for 30 minutes at room temperature and then exposed to 1-ethyl-3-(3-diethylaminopropyl)carbodiimide (EDC) and N-hydroxysuccinimide (NHS) (molar ratio 1:2) for 30 minutes to activate the carboxyl groups of MUA. Grids were then placed in a phosphate buffer solution (PBS), pH 7.4, containing 10 μg/ml streptavidin (Sigma) for 30 minutes and then rinsed with PBS. Streptavidin-coated grids were immersed in 1 M glycine for 30 minutes to quench the reaction and then rinsed with PBS. We used S. aureus isolate 43SA (isolated from a CF pig with pulmonary infection⁵), and P. aeruginosa PAO1. Molecular typing and antibiotic susceptibility profiling of S. aureus isolate 43SA was performed using standard methods. The 43SA strain is methicillin-sensitive and belongs to sequence type 398 (ST398), which is very common in pigs and often transferred to farmers ⁶. Bacteria were cultured to log phase growth and 1×10⁸ bacteria were mixed with 0.1 mg/ml N-hydroxysulfosuccinimide (sulfo-NHS)-biotin (Thermo Scientific) for 1 hour at room temperature. Streptavidin-coated grids were then incubated with biotinylated bacteria for 30 minutes and rinsed in PBS.

Bacteria-coated grids were used in three preparations. They were placed on the airway surface in vivo in pigs. They were tested in ASL removed from pigs after methacholine stimulation. They were also placed on the apical surface of primary cultures of differentiated airway epithelia. In each case, after removal from ASL, they were immediately (2-3 seconds) rinsed with PBS and then immersed in PBS containing the fluorescent indicators SYTO 9 and propidium iodide (Live/Dead BacLight™ Bacterial Viability assay, Invitrogen). Propidium iodide staining and this assay indicate the percentage of dead cells (Invitrogen, Molecular Probes product information and Fig. S7). After 15 minutes, the grids were rinsed with PBS and placed on slides for imaging with a laser scanning confocal microscope (Olympus FV1000). For each experiment, we tested bacteria-coated grids that were immersed in saline rather than exposed to ASL; the percentage of dead bacteria was 2-6%. In FIGS. 1D and 1G, the shortest exposure to ASL was 30 seconds. When bacteria-coated grids were placed on the airway surface of wild-type pigs in vivo for a shorter time (5 seconds), 15±2% (n=4) of the bacteria were dead. Of note, a study using high-speed atomic force microscopy found that antimicrobial proteins disrupted the bacterial surface at variable rates ranging from about 10 seconds to minutes'. During development of the bacteria-coated grid assay, we assessed other methods of labeling bacteria including use of bacteria expressing GFP and bacteria labeled with streptavidin-Alexa Fluor 647, other substrates including fibers and planar films, and other methods of attaching bacteria including antibody and protein A. The methods we adopted proved to be the most reliable for detecting live and dead bacteria.

For each experimental condition, time point, and/or animal, 2 or 3 grids were examined. For each grid, all live and dead bacteria were counted in 5-16 individual microscopic (60×) fields; each microscopic field contained about 100-1000 bacteria. The percentages of dead bacteria in each field were then averaged to determine the percentage of dead bacteria for the experimental data point. Operators were blinded to genotype. In some experiments, two different operators obtained similar results.

The total number of bacteria (live plus dead) attached to grids varied from experiment to experiment. For an individual grid, the number of bacteria per microscopic field also varied from field to field with an average coefficient of variation of 0.56 for grids applied to ASL in vivo and in vitro. Figure S6 shows the average number of bacteria per microscopic field for the data in FIGS. 1D and 1G. The total number of bacteria (live plus dead) in randomly selected microscopic fields did not differ between grids exposed to CF or non-CF ASL or in conditions where many or a few bacteria were killed. In addition, the total number of bacteria per microscopic field (live plus dead, mean±SD) did not differ for bacteria exposed for 30 seconds to water (232±94 total bacteria per field, 4.6% dead), saline (230±103 total bacteria per field, 4.3% dead) or 70% alcohol (228±108 total bacteria per field, 97% dead).

Bacteria-coated grids were also prepared for scanning electron microscopy using standard procedures. Briefly, the grids were fixed in 2.5% gluteraldehyde in 0.1 M cacodylate buffer followed by post fixation in 1% osmium tetroxide. The grids were then dehydrated in a graded series of ethanol, transitioned to hexamethyldisilizane, air dried overnight and mounted on aluminum stubs. Following sputter coating with gold/palladium, the samples were imaged in a Hitachi S-4800 scanning electron microscope (Pleasanton, Calif.).

### Micro-CFU and Radial Diffusion Assays

S. aureus strain 43SA and strain E. coli pCGLS-1 were used. Micro-CFU assays were performed as previously described. When ASL was collected in vivo, each micro-CFU assay contained an initial inoculum of approximately 1×10⁶ CFU/ml. When ASL was collected from cultured epithelia, each micro-CFU assay had an initial inoculum of approximately 3.3×10³ CFU/ml. Radial diffusion assays with 10 mM sodium phosphate, pH 7.4 in 1% agarose were performed with a final O.D. of bacteria of 0.02 as previously described.

### Collection of ASL for Protein and Antimicrobial Studies

ASL was collected from pigs anesthetized with ketamine (20 mg/kg, IM) and xylazine (2 mg/kg, IM), and maintained with propofol (2 mg/kg, IV). The neck was dissected to expose the trachea. Tracheal secretion was stimulated by administering methacholine (2.5 mg/kg, IV). After approximately 5 minutes, tracheal secretions were collected by making a small incision in the anterior tracheal wall and inserting a sterile polyester tipped applicator (Puritan Medical Products Co.; Guilford, Me., USA) to collect ASL. The probe was then inserted into a microcentrifuge tube and secretions were recovered by centrifugation. This procedure produced approximately 10-20 μL of ASL fluid from each animal. For assays of ASL proteins, samples were immediately placed on ice and frozen at −80° C. until use. For assays of antimicrobial activity, samples were used immediately.

ASL was also collected from primary cultures by rinsing the apical surface with 100 μH₂O. Based on our earlier wore¹⁰, we estimate that collection produced an approximate 1:80 to 1:125 dilution of ASL.

### Measurement of Amounts of ASL Antimicrobial Proteins

To immunoblot for lactoferrin, PLUNC, and SP-A, 10 μl of a 1:10 dilution of the ASL was separated on 4-15% Tris-HCl gels and transferred to PVDF membranes, followed by blocking in TBS-Tween containing 2% BSA. Membranes were incubated with a primary antibody (rabbit anti-human lactoferrin, cat#RLAC-80A, Immunology Consultants; monoclonal anti-human PLUNC, cat.# MAB1897, R&D Systems; or polyclonal antisera against porcine SP-A¹¹). Membranes were washed 4 times using TBS-Tween, then incubated with secondary antibody conjugated to horseradish peroxidase (Thermo Fisher Scientific; Rockford, Ill., USA) at a 1:20,000 dilution for 1 hour. After 5 more washes in TBS-Tween, protein bands were detected using SuperSignal West Pico Chemiluminescent Substrate (Thermo Fisher Scientific). Membranes were then exposed to film and densitometry performed. The same ASL samples were used for each western blot and stripped between westerns with Restore Western Blot Stripping Buffer (Thermo Scientific).

Lysozyme was measured using lysoplates as previously described⁹. Undiluted methacholine-stimulated secretions (5 μl) were used and compared to a standard of human lysozyme (L1667, Sigma, St. Louis, Mo.).

Collection and determination of ASL Na⁺ and K⁺ concentrations

Pigs were anesthetized with ketamine (20 mg/kg, IM) and xylazine (2 mg/kg, IM), followed by propofol (2 mg/kg, IV). The trachea was surgically exposed and accessed anteriorly using heat cautery. We then made a small anterior incision through the tracheal rings using heat cautery to prevent bleeding. To ensure that air was completely humidified the animal was studied in a humidified chamber (100% relative humidity, 25-30° C.).

ASL was collected using a procedure designed to minimize the generation of excessive capillary forces during sampling. We fused thin lens paper (VWR Scientific Products, West Chester, Pa.) with Parafilm M (Pechiney Plastic Packaging, Menasha, Wis.) in an oven (205° C.) for 70-90 seconds. This procedure further reduced the volume of liquid that the paper would absorb and minimized evaporation from the surface not touching ASL. We prepared 0.5×2 cm strips, washed them 3 times in double distilled water, and dried them overnight at 40° C.

The Parafilm-fused paper strips were weighed and then gently placed in contact with the luminal surface of the posterior trachea for 15 second. Immediately after removal from the trachea, Parafilm-fused paper strips were placed (paper side up) on a precision balance (Mettler-Toledo XP 26DR; Columbus, Ohio). Mass measurements were recorded by a synchronized computer 10 times per second for 200 seconds (BalanceLink; Mettler-Toledo; Columbus, Ohio) while evaporation occurred. The amount of ASL collected was determined by plotting mass vs. time, fitting a one-phase exponential decay to the data (GraphPad Prism 5; GraphPad Software, Inc.), and extrapolating to mass at time 0, i.e., the time at which the strip was removed from the airway surface. We then dried the strips overnight at 40° C. and measured the dry mass. 3.0±0.5 μl of ASL (n=14) was collected per cm².

To dissolve the dried ASL contents, the strips were placed in 1 ml flame photometer internal standard solution (Instrumentation Laboratory; Bedford, Mass.) overnight. We then measured Na⁺ and K⁺ content with an IL943 Flame Photometer and compared output to calibration curves of solutions containing known mole contents (Instrumentation Laboratory; Bedford, Mass.). ASL Na⁺ and K⁺ concentrations were calculated by dividing the mole content of cation by the mass of solvent. The mass of ASL solvent was determined as the difference between the initial ASL sample mass and the dry sample mass.

Na⁺ and K⁺ concentrations were measured after methacholine administration (2.5 mg/kg, IV) to stimulate submucosal gland and goblet cell secretion¹². To collect the readily visible secretions, we lightly applied a sterile polystyrene applicator (Puritan Medical Products Co.) to the tracheal surface. The applicator tip was then suspended and sealed in a microcentrifuge tube, and liquid was isolated by centrifugation through a layer of water-saturated oil. We diluted secretions 1:10 in double distilled H₂O, added 10 μl to 1 ml of flame photometer Internal Standard, measured Na⁺ and K⁺ content by flame photometry, and calculated the ion concentrations.

### Measurement of ASL pH

To assess pH in vivo, we used non-invasive dual lifetime referencing to interrogate a 3×3 mm planar optode (pH sensitive foil, PreSens GmbH, Regensburg, Germany)¹³ applied directly to the tracheal surface. The device used to transmit and receive the excitation and emission light was a single channel pH meter (pH-1 mini; PreSens GmbH). The tip of the fiberoptic pH meter was kept at the same distance from the tracheal surface in all samples and confirmed by recording the amplitude registered by the device. Calibration before each set of measurements was done by placing the planar optode on the surface of a flat filter soaked in standard pH buffers. To minimize alterations in CO₂ during placement of the probe, the experiments were done in an environment of 5% CO₂; hence the CO₂ concentration in ASL was likely>5% due to CO₂ production by the pigs. Thus, the pH values are likely lower than normally occur.

To assess pH ex vivo, secretion was stimulated with methacholine, and 5 minutes later, ASL was removed using the same methods as described for measurement of ion concentrations. Ten minutes after removal from the pig, ASL pH was measured using a needle-type Fiber Optic pH Meter (World Precisions Instruments Inc., #502123, Sarasota, Fla.). The pH meter was calibrated before each set of measurements. After removal and during measurement, the CO₂ over the sample was ambient, i.e., approximately zero, which likely accounts for the higher pH values in the ex vivo ASL.

To assess pH in primary cultures of airway epithelia, we used the fluorescent ratiometric pH indicator SNARF conjugated to dextran (Molecular Probes Inc., D-3304). SNARF was prepared as a suspension in perfluorocarbon and 200 μl was added to the apical surface. Two hours later, epithelia were studied in a humidified, 5% CO₂ atmosphere at 37° C. on the stage of an inverted laser scanning microscope (Zeiss 510 Meta NLO). SNARF was excited at 488 nm, fluorescence intensity was measured at 580 nm and 640 nm, and pH was calculated as described¹⁴.

### Methods for Changing CO₂ and for Aerosolizing HCO₃⁻

To decrease airway pH in non-CF pigs, CO₂ concentration was controlled in a humidified chamber (100% measured relative humidity, 25-30° C.) using 0 or 15% CO₂ and 21% O₂ in N₂. To increase airway pH in CF pigs, 50 μl of either 100 mM NaHCO₃ or 100 mM NaCl (as a control) were aerosolized (FMJ-250 and 1A-1C, Penn-Century, Wyndmoor, Pa.) at 20 cm over the exposed trachea; that approach achieved an average deposition of about 1 μl/cm² of fluid.

### Luminescence Antibacterial Assay

To assess the effect of pH on the antibacterial activity of lysozyme and lactoferrin, we used a luminescence assay of bacterial viability that has been described previously ¹⁵. The bacteria used were S. aureus Xen29 (Caliper LifeSciences Bioware™, Hopkinton, Mass.) and E. coli DH5a (GIBCO BRL, Life Technologies, Grand Island, N.Y.) expressing a luminescence plasmid pCGLS1. E. coli were grown in Luria-Bertani medium containing ampicillin (100 μg/ml, to maintain the plasmid) at 30° C. with shaking. For S. aureus, kanamycin 10 μg/ml was used instead of ampicillin. Log phase bacteria were centrifuged and resuspended in 1% Tryptic Soy Broth medium with 10 mM HEPES titrated to pH values from 6.8-8.0. E. coli (5×10⁶ CFU) or S. aureus (5×10⁵ CFU) were incubated with the indicated concentrations of lysozyme or lactoferrin in 96-well plates (Optiplate-96, Perkin Elmer, Waltham, Mass.) in a total volume of 120 μl. Controls were the same conditions in the same plates, but without lysozyme or lactoferrin. After incubation at 30° C. for the indicated times, luminescence was measured with a luminometer (Spectra Max L, Molecular Devices, Sunnyvale, Calif.) and was reported as relative light units (RLU). A previous study¹⁶ determined that reductions in luminescence have an excellent correlation with a decrease in CFU.

### Microarray Analysis

Trachea and bronchus tissue samples were dissected from newborn piglets within 12 hours of birth. Samples were cut into about 5 mm³ pieces and stored in RNA/ater RNA Stabilization Reagent (Ambion; Austin, Tex.) using the manufacturer's recommended protocols. Primary cultures of differentiated CF and non-CF tracheal epithelia were prepared as described above. Total RNA was isolated with TRIzol Reagent (Invitrogen; Carlsbad, Calif.). Only RNA samples attaining a minimum of 7.0 RNA Integrity Number on the Agilent 2100 Bioanalyzer (Agilent Technologies; Palo Alto, Calif.) were processed. 5 μg of total RNA was used to generate biotinylated cRNA using the Affymetrix Gene Chip one-cycle target labeling kit (Affymetrix, Inc.; Santa Clara, Calif.) according to the manufacturer's recommended protocols, and then hybridized to the Affymetrix Porcine GeneChip (23,937 probe sets that interrogate approximately 23,256 transcripts from 20,201 S. scrofa genes). Each sample hybridization (trachea, bronchus, and cultured tracheal epithelia) was performed on a separate day, with all genotypes represented in each run. The arrays were washed, stained, and scanned using the Affymetrix Model 450 Fluidics Station and Affymetrix Model 3000 scanner, and data collected using the GeneChip Operating Software (GCOS), v.1.4, using the manufacturer's recommended protocols. Partek Genomics Suite Software (Partek Inc., St. Louis, Mo.) (one-way ANOVA analysis) was used to analyze the data.

### Quantitative RT-PCR

Primers to specifically amplify porcine GAPDH, lactoferrin, lysozyme, S100A9, and PBD-2 were designed and validated using standard procedures. Total RNA was harvested using TRIzol (manufacturer's recommended protocol) from the trachea and bronchus of 6 CFTR^{+/+} and 6 CFTR^{-/-} pigs and from primary cultures of tracheal epithelia from 8 CFTR^{+/+} and 8 CFTR^{-/-} pigs. Reverse transcription with 1 μg total RNA was performed using the High-Capacity cDNA Reverse Transcription kit (Applied Biosystems). Ten nanograms of cDNA and 50 picomoles of both forward and reverse primers were used per reaction for quantitative PCR. POWER SYBR Green PCR master mix (Applied Biosystems) was used for quantification. GAPDH cDNA levels were used to normalize expression of lactoferrin, lysozyme, S100A9, and PBD-2. Fold change was generated using average ΔΔCt values for each genotype. Error bars were generated using standard error of ΔΔCt values of each sample. The primers we used were:

GAPDH
Forward:
(SEQ ID NO: 1)
GACTTCGAGCAGGAGATGG
Reverse:
(SEQ ID NO: 2)
GCACGGTGTTGGTGTTGGCGTAGAGG
Lactoferrin
Forward:
(SEQ ID NO: 3)
AGCCATCGCTACCTGAAACATGC
Reverse:
(SEQ ID NO: 4)
ATCATGAAGGCACAGGCTTCCAG
Lysozyme
Forward:
(SEQ ID NO: 5)
TGCAAAGAGGGTTGTCAGAG
Reverse:
(SEQ ID NO: 6)
AAGAGACAAGGTGAGCTGAAG
S100A9
Forward:
(SEQ ID NO: 7)
TCAGCCAGAGCCCTATAAATGCTG
Reverse:
(SEQ ID NO: 8)
TCTTCCTGCACTCTGTCCAAGC
PBD-2
Forward:
(SEQ ID NO: 9)
GG ATT AAGGGACCTGTTACAG
Reverse:
(SEQ ID NO: 10)
GCAAATACTTCACTTGGCCTG

### Statistical Analysis

Data are presented as means±standard errors of the mean (SEM). Unless otherwise indicated, statistical analysis used an unpaired t test. Differences were considered statistically significant at P<0.05.

The Supplementary Information provides full descriptions of experimental methods and procedures.

### Accession Numbers

Microarray data have been deposited in the Gene Expression Omnibus with GEO accession numbers GSE36906 and GSE21071, which are incorporated by referenced herein.

### REFERENCES

1. Welsh et al., in The Metabolic and Molecular Basis of Inherited Disease eds C. R. Scriver et al.) pg. 5121 (2001).
2. Quinton, Am. J. Physiol. Cell Physiol. 299:C1222 (2010).
3. Gibson et al., Am. J. Respir. Crit. Care Med., 168:918 (2003).
4. Wine, J. Clin. Invest., 103:309 (1999).
5. Guggino, Cell, 96:607 (1999).
6. Machen, Am. J. Physiol. Cell Physiol., 291:C218 (2006).
7. Rogers et al., Science, 321:1837 (2008).
8. Stoltz et al., Sci. Transl. Med., 2:29ra31 (2010).
9. Fischer & Widdicombe, J. Membr. Biol., 211:139 (2006).
10. Poulsen et al., Proc. Natl. Acad. Sci. U.S.A., 91:5340 (1994).
11. Smith & Welsh, J. Clin. Invest., 89:1148 (1992).
12. Chen et al., Cell, 143:911 (2010).
13. Garnett et al., J. Biol. Chem., 286:41069 (2011).
14. Grubb & Boucher, Physiol. Rev., 79:S193 (1999).
15. Foundation, C. F. Cystic Fibrosis Foundation Patient Registry: Annual Data Report 2010. (Bethesda, Md., 2010).
16. Ganz, J. Leukoc. Biol., 75:34 (2004).
17. Wine & Joo, Proc. Am. Thorac. Soc., 1:47 (2004).
18. Smith et al., Cell, 85:229 (1996).
19. Lehrer et al., Annu. Rev. Immunol., 11:105 (1993).
20. Zabner et al., Mol. Cell, 2:397 (1998).
21. Goldman et al., Cell, 88:553 (1997).
22. Matsui et al., Cell, 95:1005 (1998).
23. Tarran et al, Mol. Cell, 8: 149 (2001).
24. Knowles et al., J. Clin. Invest., 100:2588 (1997).
25. Selsted et al., Infect. Immun., 45:150 (1984).
26. Song et al., Am. J. Physiol. Cell. Physiol., 290:C741 (2006).
27. Coakley et al., Proc. Natl. Acad. Sci. U.S.A., 100:16083 (2003).
28. Laube et al., Curr. Top. Microbiol. Immunol., 306:153 (2006).
29. Do et al., J. Immunol., 181:4177 (2008).
30. Singh et al., Am. J. Physiol., 279:L799 (2000).

### SUPPLEMENTAL REFERENCES

Rogers et al., Science, 321:1837 (2008).
Rogers et al., J. Clin. Invest., 118:1571 (2008).
Karp et al., in Epithelial Cell Culture Protocols, 188:115 (ed C. Wise) (2002).
Chen et al., Cell, 143:911 (2010).
Stoltz et al., Sci. Transl. Med., 2:29ra31 (2010).
Smith et al., PLoS ONE, 4:e4258 (2009).
Fantner et al., Nat. Nanotechnol., 5:280 (2010).
Cole et al., J. Microbiol. Methods, 41:135 (2000).
Lehrer et al., J. Immunol. Methods, 137:167 (1991).
Zabner et al., Mol. Cell, 2:397 (1998).
van Eijk et al., J. Immunol., 171:1431 (2003).
Singer et al., Nat. Med., 10:193 (2004).
Blossfeld & Gansert, Plant Cell Environ., 30:176 (2007).
Shuba et al., Cell Calcium, 44:220 (2008).
Singh et al., Am. J. Physiol., 279:L799 (2000).
Travis et al., Am. J. Respir. Cell Mol. Biol., 20:872 (1999).
Sang et al., Mamm. Genome, 17:3329, (2006).

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method to prevent, inhibit or treat microbial infection in an avian or mammal, comprising:
administering to an avian or mammal having a microbial infection or suspected of being exposed to a microbe an effective amount of a composition having one or more reagents that increase pH.

2. The method of claim 1 wherein the composition is administered to an epithelial surface.

3. The method of claim 1 wherein the surface is the skin, cornea or intestinal epithelium.

4. The method of claim 1 wherein the composition is administered as an aerosol.

5. The method of claim 1 wherein the composition is administered as liquid drops.

6. The method of claim 1 wherein the composition is a cream.

7. The method of claim 1 wherein the composition is orally administered.

8. The method of claim 7 wherein the composition is a sustained release composition.

9. The method of claim 1 wherein the composition comprises a buffer.

10. The method of claim 9 wherein the buffer comprises HCO₃⁻, acetate, HEPES, or TRIS.

11. The method of claim 1 wherein the reagent inhibits inhibit proton secretion.

12. The method of claim 1 wherein the reagent inhibits. H⁺-ATPases, Na⁺—H⁺ exchangers, H⁺-conducting ion channels, or other transporters or channels.

13. The method of claim 1 wherein the one or more reagents stimulate secretion of a base.

14. The method of claim 1 wherein one reagent stimulates secretion of HCO₃⁻, stimulates channels that conduct bases, or stimulates Na⁺—HCC₃⁻ transporters.

15. The method of claim 1 wherein the one or more reagents stimulate H⁺ absorption or .inhibit HCO₃⁻ or other base absorption.

16. The method of claim 1 which employs a plurality of reagents that increase pH.

17. The method of claim 1 wherein the microbe is a gram negative bacterium.

18. The method of claim 17 wherein the microbe is a gram positive bacterium.

19. The method of claim 1 wherein the mammal is a human.

20. The method of claim 1 wherein the mammal has a defect in CFTR.
